(19) United States
(12) Patent Application Publication
(10) Pub. No.: US 2016/0175180 A1
(43) Pub. Date: Jun. 23, 2016

(54) Weight Bearing Brace Device

(71) Applicant: Reid Bond, Amarillo, TX (US)

(72) Inventor: Reid Bond, Amarillo, TX (US)

(21) Appl. No.: 14/581,382

(22) Filed: Dec. 23, 2014

(51) Int. Cl. A61H 3/00; A61F 5/01

(52) U.S. Cl. CPC A61H 3/00; A61F 5/0102

(57) ABSTRACT

A weight bearing brace device reduces the weight borne by the legs of a person. The device includes a belt, the belt is securable in a loop to be worn around a waist of a user. A pair of braces is coupled to the belt. Each of the braces is positioned on an associated side of the loop wherein each brace extends downwardly from the waist along an exterior side of an associated leg of the user. Each of a pair of soles is coupled to a distal end of an associated one of the braces wherein each sole is configured to be positioned under an associated foot of the user.

### BACKGROUND OF THE DISCLOSURE

1. Field of the Disclosure

The disclosure relates to brace devices and more particularly pertains to a new brace device for reducing the weight borne by the legs of a person.

2. Summary of the Disclosure

An embodiment of the disclosure meets the needs presented above by generally comprising a belt, the belt is securable in a loop to be worn around a waist of a user. A pair of braces is coupled to the belt. Each of the braces is positioned on an associated side of the loop wherein each brace extends downwardly from the waist along an exterior side of an associated leg of the user. Each of a pair of soles is coupled to a distal end of an associated one of the braces wherein each sole is configured to be positioned under an associated foot of the user.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of a weight bearing brace device according to an embodiment of the disclosure.

FIG. 2 is a front view of an embodiment of the disclosure.

FIG. 3 is a front view of an embodiment of the disclosure in use.

FIG. 4 is a detailed view of a fine adjustment connection of an embodiment of the disclosure.

FIG. 5 is a detailed side view of a bottom area of an embodiment of the disclosure.

FIG. 6 is a partial side view of a plurality of joints of an embodiment of the disclosure in use.

FIG. 7 is a detailed side view of a gross adjustment connection of an embodiment of the disclosure.

FIG. 8 is a detailed side view of an adjustable connection of an embodiment of the disclosure.

### DESCRIPTION OF THE PREFERRED EMBODIMENT

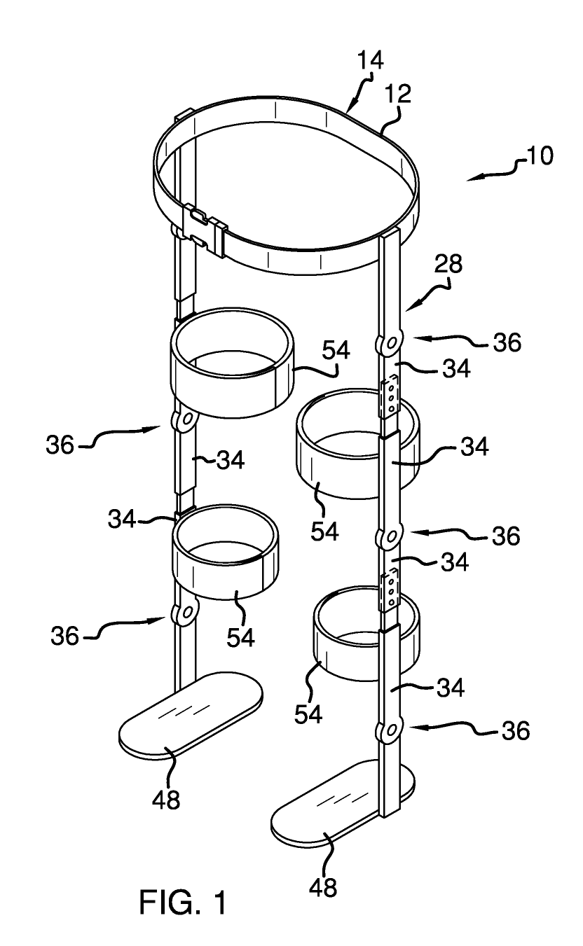

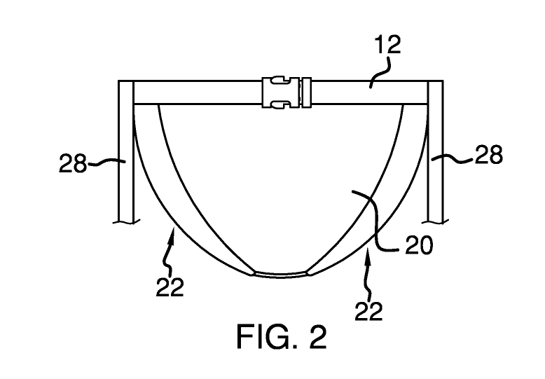

With reference now to the drawings, and in particular to FIGS. 1 through 8 thereof, a new brace device embodying the principles and concepts of an embodiment of the disclosure and generally designated by the reference numeral 10 will be described.

As best illustrated in FIGS. 1 through 8, the weight bearing brace device 10 generally comprises a belt 12 securable in a loop 14 wherein the belt 12 is configured for being worn around a waist 16 of a user 18. The belt 12 is incorporated into a garment 20 configured to be worn by the user 18. The garment 20 has a pair of openings 22 positioned below the belt 12 wherein the garment 20 is configured to receive the legs 24 of the user 18 therethrough such that the garment 20 extends under a crotch 26 of the user 18. The garment 20 may comprise underwear worn beneath an extrinsic pair of pants or a pair of pants intended to be worn over conventional underwear.

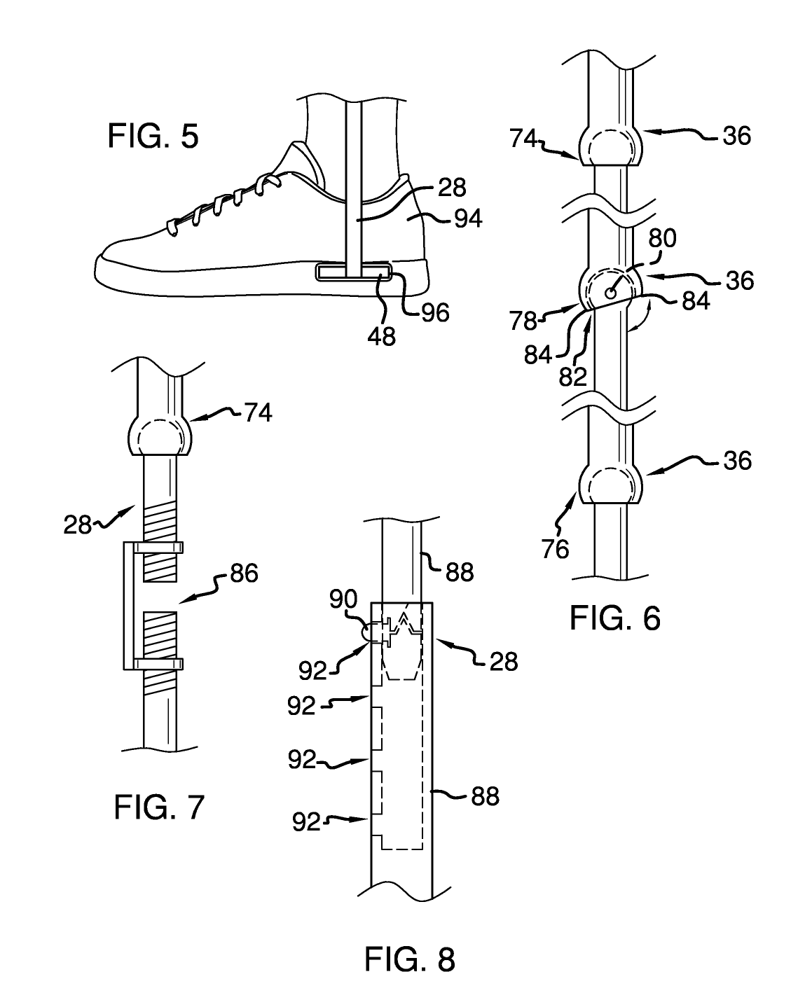

Each of a pair of braces 28 is coupled to the belt 12. Each of the braces 12 is positioned on an associated side 30 of the loop 14 wherein each brace 28 is configured to extend downwardly from the waist 16 along an exterior side 32 of an associated leg 24 of the user 18. Each brace 28 may have nested sections 34 such that the overall length of the brace 28 is adjustable. Each of a plurality of joints 36 is coupled to each brace 28 wherein each brace 28 is articulated to bend with the hip 38, knee 40, and ankle 42 of the associated leg 24 of the user 18. The nested sections 34 may be provided between the hip 38 and knee 40, the knee 40 and the ankle 42, and the ankle 42 and a bottom 44 of a foot 46 of the user 18 to substantially align the joints 36 with the hip 38, knee 40, and ankle 42. The nested sections 34 may be secured into static relative positions through conventional structures such as pins, compressions collars, or the like. Each of a pair of soles 48 is coupled to a distal end 50 of an associated one of the braces 28 wherein each sole 48 is configured to be positioned under an associated foot 46 of the user 18. The sole 48 may extend along a part of the length of the foot 46 or be substantially coextensive with the length of the foot 46. The sole 48 extends fully across the foot 46 and is shaped to conform generally to the curvature of the bottom 44 of the foot 46 to be comfortable when the foot 46 rests against the sole 48. The sole 48 is at a static substantially right angle to the brace 28 such that force exerted on a bottom surface 52 of the sole 48 is transferred upwardly through the brace 28. Each sole 48 is further configured and shaped to be positionable within a shoe worn by the user 18. Alternatively, the sole 48 may be insertable through a slot 96 in a shoe 94 as shown in FIG. 5.

As shown in FIG. 6, each joint 36 may more particularly be designed for movement related to the particular body joint adjacently positioned. A hip joint 74 and ankle joint 76 may be ball and socket type allowing for full rotation and movement while providing support. A knee joint 78 may be restricted to pivoting within a plane. Further, the knee joint 78 may incorporate a disc 80 inserted into a slit 82 such that opposite ends 84 of the slit 82 restrict the range of pivoting of the knee joint 78.

Each of a pair of sleeves 54 is coupled to an associated one of the braces 28. Each of the sleeves 54 is positioned in spaced relationship to the belt 12 wherein each sleeve 54 is configured for supporting the associated one of the braces 28 in an aligned position with the associated leg 24 of the user 18. Thus, the braces 28 support the belt 12 and the garment 20 supports the crotch 26 of the user 18 to reduce weight borne by the legs 24 of the user 18. Multiple sleeves 54 may be provided on each brace 28 to facilitate and maintain proper alignment of the brace 28 with the associated leg 24. Each sleeve may be of a flexible nature to maintain alignment with the associated leg 24 through lateral force without transferring substantial axial force to the bones of the leg 24.

In FIG. 4, adjacent sections 60 of the braces 28 may be coupled together using a threaded rod 62 and locking nuts 64 to make fine adjustments to the length of the overall brace 28. These may be used in place of or in combination with conventional telescopic adjustment methods as described above. Alternatively, as shown in FIG. 7, a turnbuckle style connection 86 may be incorporated into the brace proximate the hip of the person to provide fine adjustment to the level of the belt 12 relative to the sole 48 on each side. As seen in FIG. 8, course adjustment may be achieved using nested sections 88 and a pin 90 extending through a selectable aperture 92 in a conventional manner. The pin 90 may be biased to extend outwardly into the selected aperture 92 in a conventional manner.

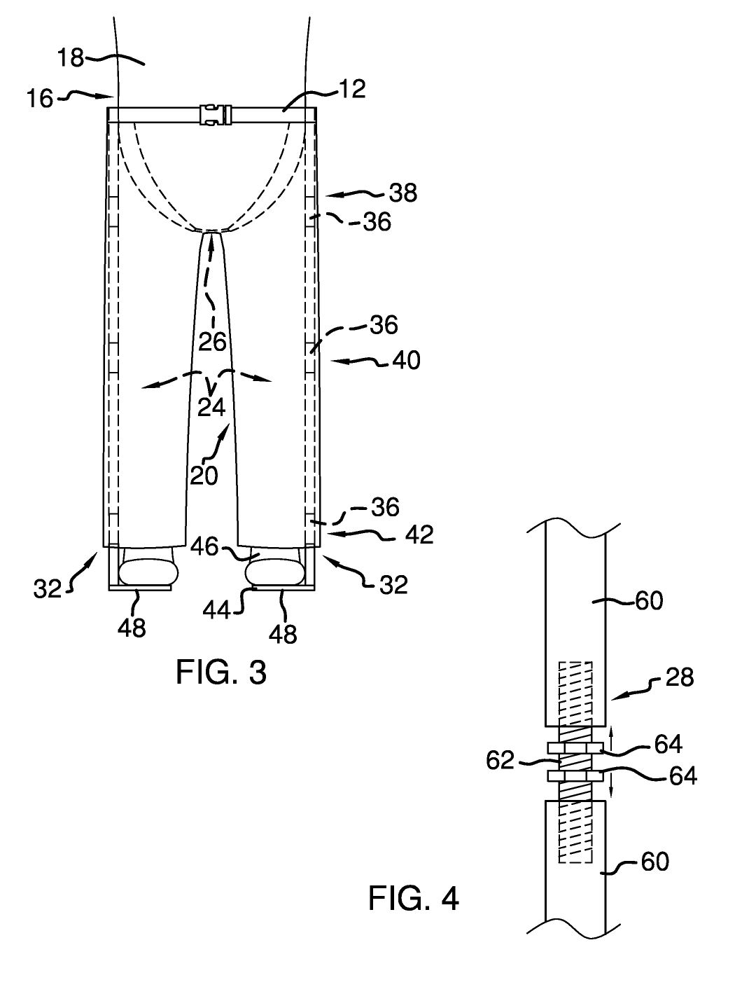

As shown in FIG. 3, the garment 20 may be a pair of pants 66 having a pair of channels 68 extending down each leg 70 of the pants 66. The braces 28 extend through respective ones of the channels 68. Openings 72 may be provided at any desired position in the channels 68 to access the braces 28 for adjustment and sizing. As also shown in

FIGS. 3 and in FIG. 5, the shoe 94 may be provided having the slot 96 extending into and across the shoe 94 such that the sole 48 occupies the slot 96 to integrate the sole into the shoe 94.

In use, the garment 20 is worn and the belt 12 secured into the loop 14 around the waist 16 of the user 18. The soles 48 are positioned under the feet 46 with the legs 24 extending through the sleeves 54. The braces 28 bear and transfer some of the weight of the user 18 to the belt 12 and the crotch 26 of the garment 20 to reduce the weight exerted on the hip 38, knee 40, ankle 42 and legs 24 generally to minimize joint pain and facilitate mobility of the user 18.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A weight bearing brace device comprising:
a belt, said belt being securable in a loop wherein said belt is configured for being worn around a waist of a user;
a pair of braces coupled to said belt, each of said braces being positioned on an associated side of said loop wherein each said brace is configured to extend downwardly from the waist along an exterior side of an associated leg of the user; and
a pair of soles, each said sole being coupled to a distal end of an associated one of said braces wherein each said sole is configured to be positioned under an associated foot of the user.

2. The device of claim 1, further comprising a respective plurality of joints coupled to each said brace wherein each said brace is articulated.

3. The device of claim 1, further comprising said belt being incorporated into a garment configured to be worn by the user.

4. The device of claim 1, further comprising each brace having an adjustable length.

5. The device of claim 1, further comprising a pair of sleeves, each of said sleeves being coupled to an associated one of said braces, each of said sleeves being positioned in spaced relationship to said belt wherein each said sleeve is configured for supporting said associated one of said braces in an aligned position with the associated leg of the user.

6. The device of claim 3, further comprising said garment having a pair of openings positioned below said belt wherein said garment is configured to receive the legs of the user therethrough such that said garment extends under and supports a crotch of the user.

7. A weight bearing brace device comprising:
a belt, said belt being securable in a loop wherein said belt is configured for being worn around a waist of a user, said belt being incorporated into a garment configured to be worn by the user, said garment having a pair of openings positioned below said belt wherein said garment is configured to receive the legs of the user therethrough such that said garment extends under a crotch of the user;
a pair of braces coupled to said belt, each of said braces being positioned on an associated side of said loop wherein each said brace is configured to extend downwardly from the waist along an exterior side of an associated leg of the user, each brace having an adjustable length;
a pair of soles, each said sole being coupled to a distal end of an associated one of said braces wherein each said sole is configured to be positioned under an associated foot of the user;
a respective plurality of joints coupled to each said brace wherein each said brace is articulated;
a pair of sleeves, each of said sleeves being coupled to an associated one of said braces, each of said sleeves being positioned in spaced relationship to said belt wherein each said sleeve is configured for supporting said associated one of said braces in an aligned position with the associated leg of the user wherein said braces support said belt and said garment supports the crotch of the user.
